(19) United States
(12) Patent Application Publication
(10) Pub. No.: US 2011/0228907 A1
(43) Pub. Date: Sep. 22, 2011

(54) HEAD AND NECK RADIATION LOCALIZATION USING ORAL APPLIANCE

(75) Inventors: Patrick James Gagnon, Vancouver, WA (US); John M. Holland, Portland, OR (US)

(73) Assignee: Oregon Health & Science University, Portland, OR (US)

(21) Appl. No.: 13/130,753

(22) PCT Filed: Nov. 27, 2009
(86) PCT No.: PCT/US2009/066017

§ 371 Date: May 23, 2011

Related U.S. Application Data
(60) Provisional application No. 61/118,139, filed on Nov. 26, 2008.

(51) Int. Cl. A61N 5/10; A61B 6/08

(52) U.S. Cl. 378/65; 378/205

(57) ABSTRACT

In various embodiments, methods, apparatuses, and systems for accurate patient positioning and motion tracking before and/or during head and neck radiation therapy, such as intensity modulated radiation therapy, are provided. In exemplary embodiments, a computing system may be endowed with one or more components of the disclosed apparatuses and/or systems and may be employed to perform one or more methods as disclosed herein. Exemplary embodiments provide head and neck radiation localization using, in part, an oral appliance.

### CROSS REFERENCE TO RELATED APPLICATIONS

The present application claims priority to U.S. Provisional Patent Application No. 61/118,139, filed Nov. 26, 2008, entitled “Oral Appliance for Head and Neck Radiation Localization and Methods Using Same,” the entire disclosure of which is hereby incorporated by reference in its entirety.

### TECHNICAL FIELD

Embodiments herein relate to the field of radiation therapy, and, more specifically, to head and neck radiation localization using, in part, an oral appliance.

### BACKGROUND

Radiation therapy provides medical benefits for the treatment of a variety of cancers. However, delivering radiation without damaging healthy tissue remains challenging. The challenges are especially difficult when trying to account for patient movement during treatment.

Head and neck malignancies make up about 4% of all cancers with an estimated 34,360 new cases, and 7,550 estimated deaths, in 2007. They have a wide range of presentation including locally confined tumors, loco-regionally advanced disease, and distant metastatic disease. They often require a multi-modality approach including surgery, chemotherapy, and radiation. The 5-year overall survival rate can be reasonable even for patients with locally advanced disease.

Techniques for delivery of radiation have changed dramatically in the past 7-10 years with movement toward intensity modulated radiation therapy (IMRT) as the standard of care for many head and neck sub-sites. IMRT allows for conformal dose distributions around the primary tumor and at-risk lymph node volumes in the neck while sparing critical structures including the spinal cord and parotid glands. This translates into safer treatments and reduced acute and permanent xerostomia for the patient, a major determinant of quality-of-life in this population.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an exemplary localization system in accordance with various embodiments;

FIG. 2 illustrates an exemplary localization system in accordance with various embodiments;

FIGS. 3A, 3B, and 3C illustrate an exemplary oral appliance including markers in accordance with various embodiments; and

FIG. 4 shows tracings of movement caused by couch shifts and the associated tracking of the localization system in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, methods, apparatuses, and systems for accurate patient positioning and motion tracking before and/or during head and neck radiation therapy, such as intensity modulated radiation therapy (IMRT), are provided. In exemplary embodiments, a computing system may be endowed with one or more components of the disclosed apparatuses and/or systems and may be employed to perform one or more methods as disclosed herein. Exemplary embodiments provide head and neck radiation localization using, in part, an oral appliance.

In an embodiment, one or more markers/transponders, such as sold by Calypso Medical, may be provided with/in an oral appliance, for example a standard sports-style mouth guard. The markers may be used for non-invasive tracking of tumors in general proximity of the mouth (head/neck). In an embodiment, the markers may be used to track head/neck movement to allow for pinpoint radiation delivery to cancer tissue.

The markers may each, when stimulated, emit a unique magnetic field or other measurable wave/emission. The markers may be stimulated by an array of a localization device placed in position over the patient. A built-in detector within the device then senses/identifies the positions of the markers. The patient may then be positioned prior to each treatment and the array may be left in-place during treatment allowing for continuous tracking of target volume motion throughout each treatment using tracking of the movement of the markers as a proxy for movement of the target volume/tissue. Should the target move out of tolerance, delivery of radiation may be stopped automatically or manually and the patient or target realigned prior to resuming treatment.

Delineation of gross tumor, suspected microscopic tumor, and normal structures is routinely performed with customized treatment plans being created for each patient based on their particular anatomy. Challenges in conforming radiation dose delivery to tumor areas or areas at risk for tumor involvement lie in target delineation and in providing adequate margins to accommodate for setup irregularities, patient motion, anatomical changes (e.g. weight loss), tumor motion, and normal tissue motion. Prior to the advent of IMRT, tight tolerances were less concerning given the large radiation fields, or ports, that were utilized. IMRT has brought an unprecedented need for millimeter level accuracy to the forefront of radiation oncology.

In IMRT, typically, a thermoplastic mask and head holder are customized for each patient to help position the patient in a reproducible manner on the treatment couch. The term “couch” refers broadly to the patient platform. Despite this custom immobilization, a significant freedom of movement within the system persists making exact reproduction of initial positioning difficult and unreliable. For instance, when a patient is secured in their mask, there is a significant amount of flexion/extension of the neck that may occur within the mask. When orthogonal images or cone-beam computed tomography (CBCT) scans are taken for image guidance, a bony landmark in the neck may be aligned accurately while areas relatively distant from this match point may not align correctly yet still be part of the overall treatment field. This misalignment may be problematic.

Nodal volumes that require radiation typically may extend from C1/C2 down to the level of the clavicular heads. These nodal volumes are treated with margins to accommodate setup irregularities but must be small (on the order of 3-5 mm) to protect surrounding normal tissues. Even small variations in setup may result in poor dosimetric coverage of these volumes while compromising more normal tissue than intended. Additionally, the spinal cord is a critical structure that must be protected in all definitive head and neck radiation cases. Spinal cord myelopathy may occur above 50 Gy, and gross tumor tissue is generally treated to doses approaching 70-75 Gy. Variations in setup put the cord at risk for inadvertent movement into the high-dose regions raising the possibility of long-term, devastating complications, including paralysis. Similar considerations regarding the parotid glands, larynx, esophagus, and pharyngeal constrictor muscles need to be taken into account as well. The best possible patient positioning to minimize relative anatomical deviations prior to each treatment is therefore a critical component to clinically effective and safe IMRT of the head and neck. Embodiments herein enable proper patient positioning.

Guidance images and/or couch shifts may be utilized to place a patient into an initial position before treatment. Initial positioning prior to each treatment does not, however, address all concerns regarding patient positioning for head and neck IMRT. A typical daily treatment utilizes 9-15 independent intensity-modulated fields arrayed around the patient. Delivering these fields takes, on average, 20 minutes per patient and sometimes longer if the setup needs to be adjusted and guidance images re-taken. Initial positioning does not provide any information regarding intra-fraction patient motion. This is a concern not only because of the long treatment times but also because of the relative discomfort of the thermoplastic mask and the severe acute morbidity induced over 6-7 weeks of radiation treatments. Lying still is difficult for even the most disciplined patients making the continuous monitoring of patient motion highly desirable but difficult to achieve. Thus, embodiments herein provide enhanced positioning methods and motion tracking during treatment.

Embodiments herein provide continuous, real-time motion tracking without the use of ionizing radiation, efficient patient setup with sub-millimeter accuracy, and decreased chance for human error from incorrect couch shifts. Embodiments may be used in positioning and tracking of head and neck cancer patients undergoing definitive radiation therapy.

Typical head and neck patient alignment relies on a customized thermoplastic mask for daily immobilization. The position is checked daily using some type of radiographic imaging modality. This daily image may then be fused to the treatment planning CT scan or a simulation radiograph. Correction then may be made to the patient's position on the table by moving the table in four degrees of freedom: left/right, superior/inferior, anterior/posterior, and rotation about the vertical axis (couch kick). In an embodiment, radiographic volumetric imaging, such as cone beam CT (CBCT), which is a CT scan obtained on the treatment couch with the patient in treatment position, may be used in conjunction with other imaging modalities discussed herein. This volumetric data provides the qualitative information for translational (left/right, superior/inferior, anterior/posterior) and rotational (around the vertical axis) couch shifts.

Rotations about the lateral and longitudinal axis cannot be made with a traditional radiation oncology couch setup. Additionally, current methods do not account for the fact that once the patient is immobilized, the patient is essentially rigid and couch shifts are applied to the entire patient and not to a specific region of anatomy.

The use of the system described herein permits correction of rotational errors around the lateral and longitudinal axes prior to masking. Such correction(s) may be made by patient manipulation/repositioning and/or by couch movements around the axes of rotation. In an embodiment, these adjustments are made in near real-time, while the patient is on the treatment couch.

In an embodiment, there is established a tight tolerance of less than one degree for the lateral and longitudinal rotation axes, although other tolerances may be established as desired such as 2, 3, 4, 5, 10, or more degrees. Results indicate that correction around these rotational axes prior to imaging improves the overall patient setup prior to masking, imaging, and ultimate couch position correction.

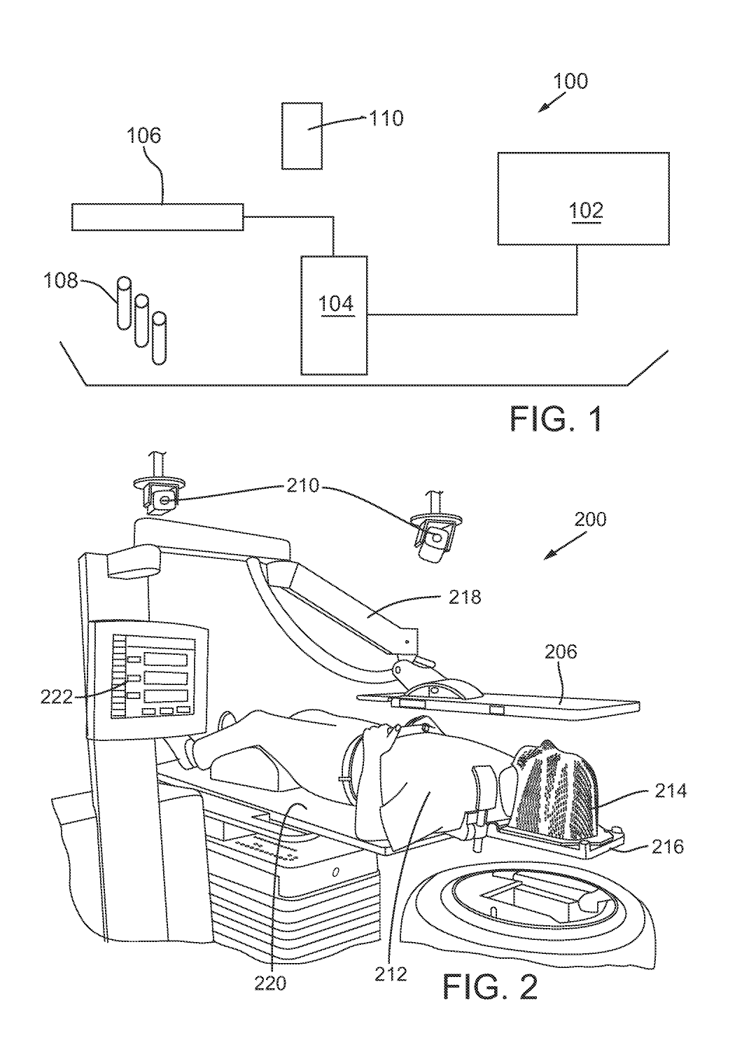

FIG. 1 illustrates an exemplary electromagnetic localization system 100 in accordance with an embodiment. System 100 includes a computing system 102 that may house hardware and/or software for treatment tracking, including providing various displays, inputs, etc. Computing system 102 may be coupled, by wire or wirelessly, to a console 104 used to position the patient and/or the radiation treatment devices. In an embodiment, computing system 102 and console 104 may be combined. Console 104 may include, or may be coupled to, an electromagnetic array 106. An electromagnetic array 106 may include an energy source to excite markers 108, and may include one or more receivers to detect transmission(s)/emission(s) from markers 108. Markers 108 may be caused to emit a magnetic field or another wave/emission that may be detected by array 106. Electromagnetic array 106 may also be positioned, in part, based on coordination with an optical system 110 including one or more cameras, such as infrared cameras. Optical system 110 may coordinate with sensors located in or on array 106.

In an alternative embodiment, markers may be configured with radio frequency transmitters. The transmitters may emit/transmit radio frequency signals that may be received by a receiver in the array or a similar device. By utilizing different signals, such as different frequencies, for each marker/transponder, or by determining the relative strength of each signal, the locations of the markers may be determined and tracked.

FIG. 2 illustrates an exemplary electromagnetic localization system 200 in accordance with an embodiment. As illustrated, system 200 includes an array 206. Array 206 may include an energy source to excite one or more markers (not shown), and may include one or more receivers to detect transmission(s)/emission(s) from the markers. An oral appliance containing the one or more markers may be placed into the mouth of patient 212. A thermoplastic mask 214 may be placed into position onto/over the face and head of patient 212. Before or after further positioning, thermoplastic mask 214 may be attached to head holder platform 216. Array 206 may be positioned over patient 212 by manipulating positioning arm 218, and based on coordination with an optical system 210 including one or more cameras, such as infrared cameras. Optical system 210 may coordinate with sensors located in or on array 206. Patient 212 is on couch 220, which may also be moved to accurately position the patient for treatment. In addition, as shown in FIG. 2, data and/or test parameters may be displayed on display 222.

In an exemplary embodiment, an oral appliance containing one or more markers may be placed into the mouth of a patient. A thermoplastic mask may be placed into position onto/over a patient's face and head but, in an embodiment, not attached to the head holder platform initially. An electromagnetic array may be moved into place in front of the patient's face and aligned based, at least in part, on images collected by one or more cameras located/mounted in the vicinity. The patient's head and neck may be adjusted on the head holder and under the mask until x, y, and z coordinates are all within a predefined threshold, such as within 0.05 cm, of treatment plan positions, relative to a reference point that represents the geographic center of the one or more markers. If not done previously, the mask may then be attached to the head holder. Treatment may then be initiated and the patient's position and motion may be continuously tracked during treatment. In an embodiment, any motion outside of a predefined tolerance may initiate a warning (such as an alarm, audible notification, or indicator light) or may automatically halt the treatment.

In an embodiment, a method for delivering radiation to a patient is provided comprising positioning the patient on a platform, the patient defining at least a lateral rotational axis and a longitudinal rotational axis; adjusting the patient along the lateral rotational axis and/or the longitudinal rotational axis; determining with a computing device a location of at least one marker, the at least one marker coupled to an oral appliance in the patient's mouth; delivering radiation to the patient; and tracking with the computing device, during radiation delivery, the location of the at least one marker.

In an embodiment, an oral appliance may be custom fit to each patient. One or more markers, such as 1, 2, 3, 4, or more, may be coupled (inserted, implanted, attached, etc.) to the oral appliance. In an embodiment, the markers may be glass markers or transponders, each containing a coiled wire such as a copper wire, which may emit or be stimulated to emit a magnetic field or other detectable wave/emission. The coordinates/locations of the markers may then be determined for example using computed tomography (CT) and such coordinates may be used for radiation treatment planning and/or targeted radiation delivery. During radiation delivery, patient movement may be continuously tracked using the markers as a tracking aid.

A suitable oral appliance may be constructed of one or more parts. In a particular embodiment, a mouth guard may be provided for engaging with the upper and/or lower teeth and maintaining the teeth/mouth in a predetermined position defined by the orientation of the mouth guard.

In embodiments, an oral appliance may be constructed from any suitable material, such as a polymeric material, for example a heat moldable material, polycaprolactone, etc. In an embodiment, the material is biocompatible.

An oral appliance may be fitted with one or more markers using a variety of methods. In one method, channels or cavities may be formed in the appliance into which the markers may be inserted. For example, cavities may be formed by drilling or boring an appliance, or by forming the cavities while molding the appliance. In another method, one or more markers may be pressed into or otherwise mixed with a moldable material prior to formation of the appliance. For example, a heat moldable material may be heated and combined with one or more markers before/during molding and formation of the appliance. In an embodiment, an appliance may be initially formed in the general shape of an oral cavity and may then be heated and placed into an individual's mouth to permit custom molding to that individual's mouth. While the appliance is in a heated and moldable state, the markers may be inserted into, such as pressed into, the moldable material.

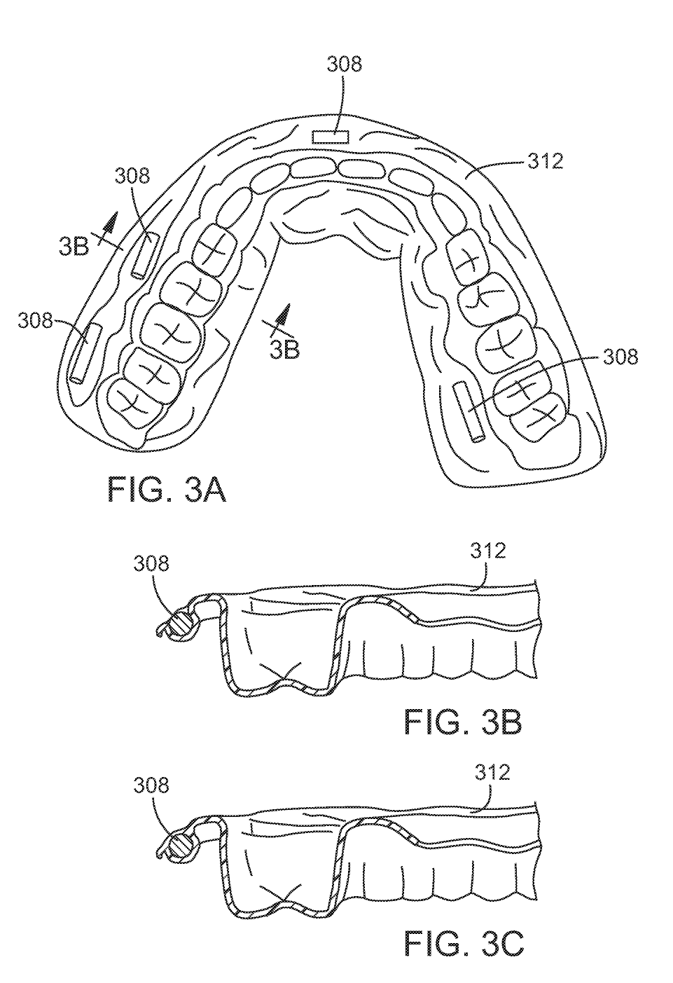

In an embodiment, markers may be placed in any suitable oral appliance to provide for tracking of head movement for targeted radiation delivery when the oral appliance is in-place in a patient's mouth. FIGS. 3A and 3B illustrate an exemplary oral appliance 312 including markers 308. Each marker/transponder 308 may be provided in any orientation with respect to oral appliance 312 and with respect to the other markers 308.

As shown in FIG. 3B, marker 308 is inserted into a channel or opening in appliance 312. As an alternative, FIG. 3C illustrates a marker 308 embedded in appliance 312.

In an embodiment, there is provided an oral appliance, comprising a biocompatible oral platform configured for insertion into a patient's mouth; and one or more electromagnetic markers coupled to the oral platform and configured to emit one or more waves in response to stimulation.

In an example, volunteers were evaluated to determine the effectiveness of embodiments described herein. Three markers (transponders) were implanted into mouth guards. The mouth guards were customized and fitted to each volunteer and thermal plastic masks were fabricated. Isocenter was set at the anterior marker position. Each volunteer was positioned on the treatment couch, the mask loosely was put in place, the electromagnetic array was positioned, the neck was flexed/extended, and the couch was moved until isocenter was within 0.5 mm in all three dimensions. The head and neck positions may be adjusted based on real time feedback from the localization system before masking. In embodiments, some adjustments may be made after masking. The mask was then secured to the head holder, and motion was tracked for five minutes. The couch was then moved 0.5 cm left, right, in, out, up, and down and these movements were compared to the tracings recorded by the localization system. Marker positions were displayed as graphic readouts on an associated monitor.

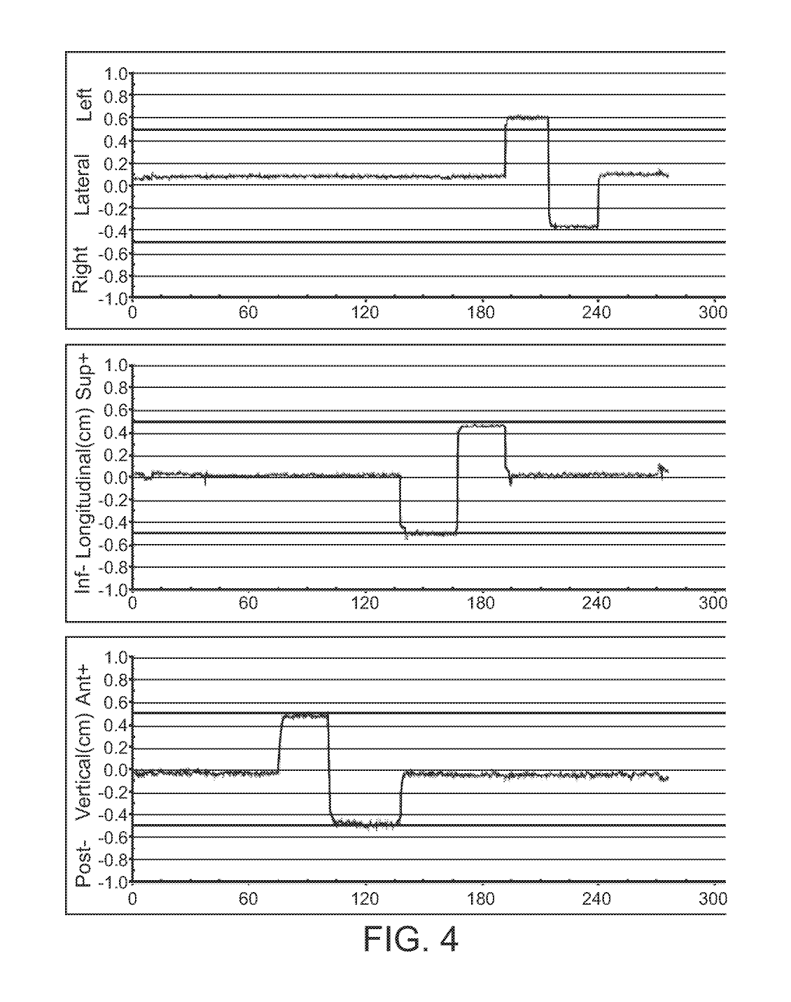

The sets of markers were localized and tracked successfully. Adjustment of the neck position prior to masking was recorded by the localization system. Motion-tracking data revealed a >2 mm deviation for only 8% of total tracking time. Deviations of 1-2 mm were recorded for 30% of total tracking time. The localization system accurately tracked all table deviations to within 0.5 mm. In addition, masking did not force the transponders out of tolerance (1 mm). See FIG. 4 for results. In FIG. 4, time is reported in seconds on the x-axis and deviations in centimeters on the y-axis. Lateral shifts are shown in the top tracing, longitudinal shifts in the middle tracing, and vertical shifts in the bottom tracing.

As shown by the above example, the localization system provides an accurate method of continuous intra-fractional monitoring of patient movement not possible with traditional imaging techniques that rely on ionizing radiation. The described approach demonstrates that this system is feasible for use in head and neck IMRT patients. Changes to neck position prior to securing the mask were successfully tracked with this system as well. This capability may also prove useful in providing accurate daily head and neck IMRT patient setup.

In an alternative example, a method may be implemented for participants with a squamous cell carcinoma of the head and neck who have been indicated for definitive radiation. A customized mouth guard may be constructed and three markers implanted into it. In this regard, a full dental evaluation may be performed and any necessary extractions performed. Stone impressions may be taken of the patient's upper and lower jaws. Using the stone impression of the upper jaw, a customized mouth guard may be fabricated using a vacuum-assisted thermoplastic polymer setup allowing for a thin and rigid mouth guard to be created.

The markers may then be fixed to the inside surface of the mouth guard paying attention to the orientation of the markers. The first marker may be placed just posterior to the central incisors, the second marker may be placed just medial to the patient's right pre-molar, 1^{st} molar, or 2^{nd} molar, and the third marker may be placed just medial to the patient's left pre-molar, 1^{st} molar, or 2^{nd} molar. The markers should generally be placed to avoid as much metal dental filling material as possible. This makes subsequent identification of the markers by computed tomography (CT) more reliable and minimizes the risk of interference with the positioning system by metal material in the teeth. Fixation may then be done with hot wax to secure the markers' positions.

The mouth guard with attached markers may then be placed back on the stone impression and another sheet of thermoplastic material may be applied using the vacuum assisted setup once more. The edges of the two thermoplastic layers may then be pinched together creating a seal that effectively sandwiches the three markers between these layers providing a very stable and reproducible setup.

The mouth guard may then be fitted to the patient's upper jaw prior to CT simulation. CT simulation is carried out, including fabrication of a thermoplastic mask and head holder system as well as reconstruction of the CT images into a 3 mm data set as well as a 1 mm data set. All images are transferred to a commercially available treatment planning system. The 1 mm slice reconstruction may then be used to determine the 3D positional coordinate locations of the markers. These coordinates are then input into the system.

In an embodiment, patients may have their custom head holder and mask molded with the mouth guard in place prior to a CT scan. Patients may then receive fractionated IMRT. Patients may receive, for example, 35 total treatments at a dose of approximately 70 Gy, although adjustments made be made or an alternative number of treatments and/or doses may be provided. For each treatment fraction, the patient may insert the mouth guard, be positioned on the head holder, loosely placed in the mask, and have the electromagnetic array of a localization system brought into position. Every other treatment may include positioning with the localization system prior to fastening of the mask to the table.

In embodiments, after the customized mouth guard with the markers is put in place, the mask may be put into place and the patient aligned by laser to points on or affixed to the mask at the time of simulation. The mask may then be released from the head holder and the array may be brought into place over isocenter. The array may be aligned by laser. The patient may then be called up on the computing system and localization may be performed. The tolerance for translations may be set at 1 cm, for example, and when achieved, the tracking option may be selected.

If rotational alignment is out-of-tolerance, which is defined to be one degree in this example, an error message appears that gives the magnitude and axis of each rotation that is out of tolerance. The patient's head may then be rotated under the loose mask to correct for these errors and the mask may then be fixed in place.

The patient may then be re-loaded in the system and localized again. Tracking may then be selected. If rotations are still greater than 1 degree off expected, another error message may appear. Re-alignment steps may then be performed as necessary until the lateral and longitudinal axes of rotation are correct to within one degree, or another threshold tolerance as desired.

Treatments may be performed under daily image guidance utilizing kV orthogonal pairs or cone-beam CT scans (CBCT). Images may be evaluated pre-treatment by a radiation oncologist. Appropriate shifts of the treatment couch may then be performed. Treatment may then occur with continuous motion tracking throughout. All kV and CBCT images may be sent to an online or offline system where shifts may be made by the physician.

In an additional embodiment, an article of manufacture is provided including a computer-readable medium having instructions stored thereon that, in response to execution by a computing device, cause the computing device to perform a method comprising determining whether a current position of a patient is equal to or less than a predefined tolerance, the patient defining at least a lateral rotational axis and a longitudinal rotational axis, the predefined tolerance defining an acceptable out of rotation alignment along the lateral rotational axis and/or the longitudinal rotational axis; determining a location of at least one marker, the at least one marker coupled to an oral appliance in the patient's mouth; and tracking with the computing device, during delivery of radiation, the location of the at least one marker.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method for delivering radiation to a patient, comprising:
positioning the patient on a platform, the patient defining at least a lateral rotational axis and a longitudinal rotational axis;
adjusting the patient along the lateral rotational axis and/or the longitudinal rotational axis;
determining with a computing device a location of at least one marker, the at least one marker coupled to an oral appliance in the patient's mouth;
delivering radiation to the patient; and
tracking with the computing device, during radiation delivery, the location of the at least one marker.

2. The method of claim 1, wherein delivering radiation to the patient comprises delivering radiation to tissue in the patient's head and/or neck.

3. The method of claim 1, further comprising fitting a mask to the patient's head and/or face.

4. The method of claim 4, wherein fitting the mask to the patient's head and/or face comprises fitting the mask to the patient's head and/or face after adjusting the patient along the lateral rotational axis and/or the longitudinal rotational axis.

5. The method of claim 4, further comprising securing the mask to the patient platform.

6. The method of claim 5, wherein securing the mask to the patient platform comprises securing the mask to a head holder integral with or coupled to the patient platform.

7. The method of claim 1, further comprising obtaining one or more radiographic images and adjusting at least one of the patient and the patient platform in accordance with the radiographic images.

8. The method of claim 7, wherein obtaining one or more radiographic images comprises obtaining one or more radiographic images using cone-beam computed tomography.

9. The method of claim 7, wherein adjusting at least one of the patient and the patient platform in accordance with the radiographic images comprises adjusting at least one of the patient and the patient platform translationally or rotationally about a vertical axis.

10. The method of claim 1, wherein positioning the patient on a platform comprises iteratively positioning the patient and determining with an associated localization system whether a current patient position is equal to or less than a predefined tolerance.

11. The method of claim 10, wherein the predefined tolerance is one degree out of rotational alignment.

12. The method of claim 1, wherein positioning the patient on a platform comprises repositioning the patient and/or the patient platform by movements around the axes of rotation.

13. An article of manufacture including a computer-readable medium having instructions stored thereon that, in response to execution by a computing device, cause the computing device to perform a method comprising:
determining whether a current position of a patient is equal to or less than a predefined tolerance, the patient defining at least a lateral rotational axis and a longitudinal rotational axis, the predefined tolerance defining an acceptable out of rotation alignment along the lateral rotational axis and/or the longitudinal rotational axis;
determining a location of at least one marker, the at least one marker coupled to an oral appliance in the patient's mouth; and
tracking with the computing device, during delivery of radiation, the location of the at least one marker.

14. The article of manufacture of claim 13, wherein the predefined tolerance is one degree out of rotational alignment.

15. An oral appliance, comprising:
a biocompatible oral platform configured for insertion into a patient's mouth; and
one or more electromagnetic markers coupled to the oral platform and configured to emit one or more waves in response to stimulation.

16. The oral appliance of claim 15, wherein the one or more electromagnetic markers are configured to emit waves in response to an external stimulation.

17. The oral appliance of claim 15, wherein the one or more electromagnetic markers are resident in channels in the oral appliance.

18. The oral appliance of claim 15, wherein the one or more electromagnetic markers are embedded in or between one or more material layers of the oral appliance.
